(19) United States
(12) Patent Application Publication
(10) Pub. No.: US 2011/0171202 A1
(43) Pub. Date: Jul. 14, 2011

(54) METHOD OF TREATING HEARING LOSS USING XIAP

(75) Inventor: Manohar Bance, Halifax (CA)

(73) Assignee: Dianovix Inc.

(21) Appl. No.: 13/000,551

(22) PCT Filed: Jul. 2, 2009
(86) PCT No.: PCT/CA09/00921

§ 371 Date: Mar. 30, 2011

Related U.S. Application Data
(60) Provisional application No. 61/078,104, filed on Jul. 3, 2008.

(51) Int. Cl. A61K 48/00; A61K 38/48; A61P 27/16

(52) U.S. Cl. 424/94.67; 514/44 R; 424/94.63

(57) ABSTRACT

Disclosed is a method of treating or preventing hearing loss in a subject. The method comprises administering to the subject in need thereof, an adeno-associated viral expression vector encoding X-linked inhibitor of apoptosis protein (XIAP). The XIAP is positioned in the vector for expression in an inner ear organ, or associated neural structures, of the subject so as to treat or prevent the hearing loss. Also disclosed is a method of treating or preventing impaired balance.

### FIELD OF THE INVENTION

The invention relates to hearing loss and more particularly to a method of treating or preventing age-related hearing loss using gene therapy.

### BACKGROUND OF THE INVENTION

Age-related hearing loss (AHL), or presbycusis, is a common neurodegenerative disorder in aged adults, which affects approximately 40% of the population by the age of 65 (NIDCD, 1995). The process of aging interacts with many other factors, such as noise exposure and miscellaneous ototoxic insults which are hazardous to the receptor hair cells (HC) and the spiral ganglion neurons (SGNs) in the cochlea. In many cases, it is difficult to distinguish between the effects of aging per se and the effects of other hazardous factors on cell death in the cochlea. Permanent hearing loss resulting from the loss of HCs and SGNs is irreversible because the cells are terminally developed and cannot be replaced by mitosis. Although great efforts have been and continue to be made to regenerate lost hair cells and SGNs in mammals, these efforts have been largely unsuccessful.

A large body of evidence implicates apoptosis in aging associated cochlea cell death or damage. During the process of aging, apoptosis can be triggered by many different factors that result in caspase activation (Spicer and Schulte, 2002). Activation of these proteases in the cochlea causes the death of HCs and SGNs (Zheng et al., 1998). Caspase inhibitors such as z-DEVD-fmk and z-LEHD-fmk have been shown to protect cochlea hair cells from cisplatin-induced death. Direct caspase inhibitor application in the inner ear also greatly enhances vestibular hair cell survival after an aminoglycoside treatment (Matsui et al., 2003). Other methods shown to partially prevent ototoxin-induced hair cell loss include the use of minocycline (Wei et al., 2005), neurotrophins (Zheng et al., 1995; Ernfors et al., 1996; Ding et al., 1999a), calpain inhibitors (Wang et al., 1999) and antioxidant therapy (Garetz et al., 1994; Lautermann et al., 1995; Ohinata et al., 2003). A common feature in these treatments is that they all block apoptosis. Unfortunately, the short duration of action of these treatments limits their utility in the treatment of presbycusis.

Members of the inhibitor of apoptosis proteins (IAP) such as X-linked IAP (XIAP), human-IAP1 (HIAP1) and human-IAP2 (HIAP2) inhibit both apoptosis initiators (e.g., caspase-9 by XIAP; caspase-8 by HIAP1 and HIAP2) and apoptosis effectors (caspases-3 and -7 by XIAP) by triggering ubiquitin-mediated degradation of these (Deveraux et al., 1997; Roy et al., 1997; Deveraux et al., 1998; Suzuki et al., 2001). As a result, manipulations that increase IAP expression increase the survival of multiple cell types in response to a variety of apoptotic triggers (e.g., (Liston et al., 1996; Robertson et al., 2000)). For example, virally mediated over-expression of XIAP reduces the loss of CA1 hippocampal neurons and preserves spatial navigation memory after transient forebrain ischemia (Xu et al., 1999) and also delays the death of cultured cerebellar granule neurons following potassium withdrawal (Simons et al., 1999). In hepatocytes, over-expression of HIAP2 inhibits the apoptosis induced by various cytokines (Schoemaker et al., 2002). Blocking caspase activity by IAP over-expression has at least two advantages over the use of exogenous inhibitors. Firstly, virally-mediated IAP expression in the inner ear produces prolonged caspase inhibition (Cooper et al., 2006; Chan et al., 2007), much longer than the duration of inhibition produced by exogenous, small molecule inhibitors. Secondly, XIAP also blocks non-caspase mediated cell death, such as that produced by activation of the c-Jun terminal kinase pathway. This makes XIAP the most potent of all known inhibitors of apoptosis (Deveraux and Reed, 1999; Deveraux et al., 1999a; Kaur et al., 2005). Another advantage over small molecule inhibitors of caspases such as zVAD-fmk or DEVD-fmk is that these are not specific for caspases, they also inhibit other cysteinyl proteases such as calpains and cathepsins (Schotte et al., 1999) and therefore, could interfere with the other cellular functions of these proteases.

XIAP is the prototypical IAP characterized by three baculoviral IAP repeats (BIRs) and the ring zinc finger motif. XIAP has been shown to bind and inhibit caspases-3, -7 and -9 (Deveraux et al., 1997; Roy et al., 1997; Deveraux et al., 1998; Takahashi et al., 1998; Sanna et al., 2002b; Sanna et al., 2002a). XIAP contains three BIR domains. BIR1 and BIR2 are located towards the N-terminus of XIAP and are sufficient to protect cells from Fas-induced apoptosis by binding to caspase-3 and -7. However, the degree of protection is less than that provided by full length XIAP. BIR3 is close to the RING domain that is located towards the c-terminus of XIAP (Deveraux et al., 1999b). It has been found that BIR3 alone is sufficient to inhibit the apoptotic initiator caspase-9 (Takahashi et al., 1998; Sun et al., 1999). XIAP binding to pro-caspases-3 and -7 prevents these proteins from being activated by caspase-8. XIAP can also directly inhibit activation of caspases-3 and -7, and accelerate the degradation of these proteins (Deveraux and Reed, 1999; Suzuki et al., 2001). In the intrinsic pathway, XIAP prevents activation of the initiator caspase-9 by cytochrome c. Moreover, XIAP blocks the feedback activation of caspases-8 and -9 by activate caspase-3. From this evidence, we can infer that XIAP is able to block or reduce apoptosis occurring through both the extrinsic and extrinsic pathways.

During recent years, XIAP based gene therapy has been evaluated in many different settings. For example, over-expression of XIAP through genetic manipulation has been demonstrated to be sufficient to prevent neuronal death in models of stroke and Parkinson's disease (Xu et al., 1999; Crocker et al., 2003; Trapp et al., 2003). The survival advantage offered by XIAP over-expression is conferred by inhibition of at least two cell death pathways: inhibition of caspase-3 (Deveraux and Reed, 1999) and c-Jun N-terminal kinase (JNK) (Igaki et al., 2002).

However, despite the aforesaid advances, there is still a need for improved methods to treat age-related hearing loss.

### BRIEF SUMMARY

In one aspect, there is provided a method of treating or preventing hearing loss in a subject, the method comprising: administering to the subject in need thereof, an adeno-associated viral expression vector encoding X-linked inhibitor of apoptosis protein (XIAP), the XIAP being positioned in the vector for expression in an inner ear organ, or associated neural structures, of the subject so as to treat or prevent the hearing loss.

In another aspect, there is provided a method of treating or preventing age-related hearing loss in a subject, the method comprising: administering to the subject in need thereof, an adeno-associated viral expression vector encoding X-linked inhibitor of apoptosis protein (XIAP), the XIAP being positioned in the vector for expression in an inner ear organ, or associated neural structures, of the subject so as to treat or prevent the hearing loss.

In still another aspect, there is provided a method of slowing the development of age-related hearing loss in a subject, the method comprising: administering to the subject in need thereof, an adeno-associated viral expression vector encoding X-linked inhibitor of apoptosis protein (XIAP), the XIAP being positioned in the vector for expression in an inner ear organ, or associated neural structures, of the subject so as to slow the development of the hearing loss.

In yet another aspect, there is provided a method of reducing inner ear cell loss, the method comprising: contacting the inner ear cell with an adeno-associated viral expression vector encoding X-linked inhibitor of apoptosis protein (XIAP), the XIAP being positioned in the vector for expression in the inner ear cell, or associated neural structures, so as to reduce loss of the inner ear cell or associated neural structures.

In yet another aspect, there is provided a method of reducing hair cell loss, the method comprising: contacting the hair cell with an adeno-associated viral expression vector encoding X-linked inhibitor of apoptosis protein (XIAP), the XIAP being positioned in the vector for expression in the hair cell cell, or associated neural structures, so as to reduce loss of the hair cell or associated neural structures.

In another aspect, there is provided a method of reducing spiral ganglion neuron loss, the method comprising: contacting the spiral ganglion neuron with an adeno-associated viral expression vector encoding X-linked inhibitor of apoptosis protein (XIAP), the XIAP being positioned in the vector for expression in the spiral ganglion neuron so as to reduce loss thereof.

In still another aspect, there is provided a method of reducing high-frequency hearing loss in a subject, the method comprising: administering to the subject in need thereof, an adeno-associated viral expression vector encoding X-linked inhibitor of apoptosis protein (XIAP), the XIAP being positioned in the vector for expression in an inner ear organ of the subject so as to treat the high frequency hearing loss.

In one aspect, there is provided a method of treating or preventing vestibular organ degeneration in a subject, the method comprising: administering to the subject in need thereof an adeno-associated viral expression vector encoding X-linked inhibitor of apoptosis protein (XIAP), the XIAP being positioned in the vector for expression in a cell of the vestibular organ so as to treat or prevent the vestibular organ degeneration

In another aspect, there is provided a method of treating or preventing impaired balance in a subject, the method comprising: administering to the subject in need thereof an adeno-associated viral expression vector encoding X-linked inhibitor of apoptosis protein (XIAP), the XIAP being positioned in the vector for expression in a cell of the vestibular organ so as to treat or prevent the impaired balance.

In still another aspect, there is provided a method of slowing the development of impaired balance in a subject, the method comprising: administering to the subject in need thereof an adeno-associated viral expression vector encoding X-linked inhibitor of apoptosis protein (XIAP), the XIAP being positioned in the vector for expression in a cell of the vestibular organ so as to slow the development of the impaired balance.

In one aspect, there is provided a pharmaceutical composition comprising: an amount of an AAV comprising a transgene positioned for expression of XIAP in an inner ear organ, or associated neural structures; and a pharmaceutically acceptable vehicle.

In an alternative aspect, there is provided use of an adeno-associated viral expression vector encoding X-linked inhibitor of apoptosis protein (XIAP), the XIAP being positioned in the vector for expression in an inner ear organ, or associated neural structures, in the manufacture of a medicament for treating or preventing hearing loss in a subject.

In another alternative aspect, there is provided use of an adeno-associated viral expression vector encoding X-linked inhibitor of apoptosis protein (XIAP), the XIAP being positioned in the vector for expression in an inner ear organ, or associated neural structures, in the manufacture of a medicament for treating or preventing age related hearing loss in a subject.

In yet another alternative aspect there is provided use of an adeno-associated viral expression vector encoding X-linked inhibitor of apoptosis protein (XIAP), the XIAP being positioned in the vector for expression in an inner ear organ, or associated neural structures, in the manufacture of a medicament for slowing the development of age-related hearing loss in a subject.

In another alternative aspect, there is provided use of an adeno-associated viral expression vector encoding X-linked inhibitor of apoptosis protein (XIAP), the XIAP being positioned in the vector for expression in an inner ear organ, in the manufacture of a medicament for reducing high-frequency hearing loss in a subject.

In another alternative aspect, there is provided use of an adeno-associated viral expression vector encoding X-linked inhibitor of apoptosis protein (XIAP), the XIAP being positioned in the vector for expression in a cell of the vestibular organ, in the manufacture of a medicament for treating or preventing vestibular organ degeneration in a subject.

In another alternative aspect, there is provided use of an adeno-associated viral expression vector encoding X-linked inhibitor of apoptosis protein (XIAP), the XIAP being positioned in the vector for expression in a cell of the vestibular organ, in the manufacture of a medicament for treating or preventing impaired balance in a subject.

In yet another alternative aspect, there is provided use of an adeno-associated viral expression vector encoding X-linked inhibitor of apoptosis protein (XIAP), the XIAP being positioned in the vector for expression in a cell of the vestibular organ, in the manufacture of a medicament for slowing the development of impaired balance in a subject.

In one aspect, there is provided a method of delivering a viral vector to an inner ear, the method comprising: partially digesting the round window membrane using a collagenase enzyme; and contacting the partially digested round window membrane with an adeno-associated viral expression vector so as to transfect the inner ear cells or associated neural structures.

In the above, the adeno-associated viral expression vector is selected from AAV1, AAV2, AAV3, AAV4, AAV5, AAV6 and AAV7. In one example, the adeno-associated viral expression vector is AAV2. In another example, the adeno-associated viral expression vector is a modified serotype-2 or -8 AAV vector. The XIAP is full length human XIAP. The hearing loss is high-frequency hearing loss. In one example, the high-frequency hearing loss is at 2 kHz and above. The inner ear organ includes the inner ear hair cell and the outer ear hair cell. The inner ear cell is a hair cell, a supporting cell, inner ear mechanical structure or a spiral ganglion neuron. The inner ear cell associated neural structures are the afferent and efferent neural processes, both of which contact or influence the inner ear hair cell function and transmit hair cell activity centrally to the brain, or from the brain to the inner ear. The hearing loss is the result of hair cell degeneration in the cochlea, or loss of supporting mechanisms that allow the hair cell to function. The hearing loss is the result of spiral ganglion neuron degeneration in the cochlea. The hearing loss is age-related hearing loss. The hearing loss is presbycusis. The impaired balance is in a subject who is aging. The vestibular organ degeneration is due to ototoxicity, viral infections of the inner ear, autoimmune inner ear diseases, genetic vestibular losses, inner ear barotraumas; or physical trauma, or surgical trauma. The vector is administered through the round window membrane. An ubiquitin promoter is used to drive expression of XIAP in cochlea cells. The hearing loss is due to ototoxicity, noise induced hearing loss, viral infections of the inner ear, autoimmune inner ear diseases, genetic hearing losses, inner ear barotrauma; physical trauma, or surgical trauma; or inflammation.

### BRIEF DESCRIPTION OF THE DRAWINGS

In order that the invention may be readily understood, embodiments of the invention are illustrated by way of example in the accompanying drawings.

FIG. 1 illustrates ABR threshold audiograms in young (2-6 month old) WT and TG mice. A: ABR audiograms for TG and WT mice at months of age; B: ABR audiograms for TG and WT mice at 6 months of age. C: Aging-related hearing loss at 2 and 6 months of age in WT littermates. D: Aging-related hearing loss at 2 and 6 months of age in TG mice. Each circle represents mean±SEM of 15-17 animals. Asterisks in A and B indicate the frequencies at which the differences were statistically significant between the two groups, p<0.05.

FIG. 2 illustrates ABR threshold audiograms at 10-14 months in WT and TG mice. A: At 10 months hearing thresholds across all frequencies were superior for TG relative to WT mice. B: At 12 months, hearing thresholds for WT and TG mice are similar at low frequencies (2, 4 and 8 kHz). C: At 14 months of age, the averaged ABR thresholds were similar for WT and TG mice at the three low frequencies (2, 4 and 8 kHz) tested while TG mice still displayed superior ABR sensitivities at the higher frequencies. In B and C, asterisks indicate the frequencies at which TG mice displayed superior ABRs (p<0.05). Each circle represents the mean±SEM of 15-17 animals.

FIG. 3 illustrates ABR threshold audiograms showing a comparison of aging-related hearing loss in WT littermates and TG mice. A: The ABR thresholds were averaged into two distinct frequency segments: 2-8 kHz as the low-frequency (LF) region (solid symbols) and 16-64 kHz as the high-frequency (HF) region (open symbols). B: The ABR-threshold audiogram from WT group at 6 months (dashed line, open circle) is compared with that from TG group at 14 months (solid line, solid circle). In A, each point represents the mean of 15-17 animals. In B, each circle represents the mean±SEM of 15-17 animals.

FIG. 4 are graphs showing hair cell loss as a percentage for both TG and WT mice (n=19 per group). Filled and open circles represent the mean±SEM for hair cell loss in TG mice and WT littermates, respectively.

FIG. 5 are representative hair cell loss images from one TG cochlea (Left) and one WT cochlea. The samples were treated with SDH staining. The images were taken from matched spots in the basal turns of the two cochleae. “Basal-1” is about 0.5 mm to the basal end of the cochlea (92% from the apex), while “basal-2” is located 1.2 mm from the basal end.

FIG. 6 is a representative Western blot showing Myc-XIAP and endo-XIAP levels in the ear and brain (temporal lobe) of 2 and 14 month old WT and TG animals. Endo-XIAP levels were higher in the ear than brain, particularly in older age mice at 14 months (14 mo).

FIG. 7 is a histogram showing the impact of all three factors (genotype, tissue and age) on the levels of endo-XIAP. Bars represent mean±SEM. Endogenous-XIAP levels were found to be higher in ears than in brains in both genotypes at 14 months compared to 2 months of age. The difference was statistically significant at 14 months of age. *p<0.05 relative to brain.

FIG. 8 is a histogram showing quantification of Myc-XIAP levels in brain and ears at 2 and 14 months of age. Each bar represents the mean±SEM. At both ages, Myc-XIAP was significantly higher in brain than ear (p<0.05).

FIG. 9 is a TEM image of a cross section from a normal round window membrane.

FIG. 10 is a photograph showing transfection of inner ear cells seen in surface preparation. GFP positive cells were seen in IHC region, not in the OHC region.

FIG. 11 are SEM images of RWM surface facing middle ear.

FIG. 12 shows the TEM images of damaged RWM at the surface to middle ear, showing damaged cells.

FIG. 13 is a TEM image of RWM immediately after the digestion showing damage to the epithelia cell.

FIG. 14 are TEM (left) and SEM (right) images from RWM 3 weeks after the digestion treatment.

Further details of the invention and its advantages will be apparent from the detailed description included below.

### DETAILED DESCRIPTION

In the following description of the embodiments, references to the accompanying drawings are by way of illustration of an example by which the invention may be practiced. It will be understood that other embodiments may be made without departing from the scope of the invention disclosed.

Unless otherwise specified, the following definitions apply:

The singular forms “a”, “an” and “the” include corresponding plural references unless the context clearly dictates otherwise.

As used herein, the term “comprising” is intended to mean that the list of elements following the word “comprising” are required or mandatory but that other elements are optional and may or may not be present.

As used herein, the term “consisting of” is intended to mean including and limited to whatever follows the phrase “consisting of”. Thus, the phrase “consisting of” indicates that the listed elements are required or mandatory and that no other elements may be present.

As used herein, the term “subject” or “patient” is intended to mean humans and non-human mammals such as primates, cats, dogs, swine, cattle, sheep, goats, horses, rabbits, rats, mice and the like.

As used herein, the term “associated neural structures” when used in conjunction with the inner ear cell associated neural structures, is intended to mean the neural processes, both efferent and afferent that contact or influence the inner ear hair cell function and transmit hair cell activity centrally to the brain, or from the brain to the inner ear.

As used herein, the term “XIAP” is X-linked inhibitor of apoptosis protein and is intended to mean any polypeptide having the activity of full-length human XIAP protein. This activity is characterized by inhibition of apoptosis and/or binding caspase 3. Examples of XIAP includes full length XIAP, including human XIAP (e.g., genbank accession numbers aac50373, cab95312, aah32729, np.sub.—001158, aaw62257, aac50518, aax29953, Q9R016, aah71665, and cai42584), and XIAP xenologues. Examples of XIAP xenologues are mouse XIAP (e.g., genbank accession numbers q60989 and np.sub.—033818), rat XIAP (e.g., genbank accession numbers aag22969, aag41193, and aag41192), domestic cow (e.g., genbank accession numbers xp.sub.—583068 and np.sub.—001030370), zebrafish (e.g., genbank accession numbers np.sub.—919377, aah55246, and xp.sub.—689837), chimpanzee (e.g., genbank accession number xp.sub.—529138), dog (e.g., genbank accession number abb03778), chicken (e.g., genbank accession number np.sub.—989919), frog (e.g., genbank accession number np.sub.—001025583 and bad98268), orangutan (e.g., genbank accession number cah91479), and catfish (e.g., genbank accession number aax35535).

The term “XIAP” also means any functional XIAP fragment, or any fusion of functional XIAP fragments. Examples of these fragments include those that consist of, consist essentially of, or include (i) BIRs 1-3, (ii) BIR3 and the RZF, (iii) BIR 3 (or a conformationally stabilized BIR of Ts-IAP, TIAP, hILP-2, or birc8), (iv) BIR2-3, (v) BIR2 and the RZF, (vi) BIR1-2, or (vii) BIR2 alone. Furthermore, “XIAP” embraces any of these fragments having an additional amino terminal methionine.

The term “XIAP” also means any fusion of full length XIAP, or a functional fragment thereof, with another polypeptide. These fusions include, but are not limited to, GST-XIAP, HA tagged XIAP, or Flag tagged XIAP. These additional polypeptides may be linked to the N-terminus and/or C-terminus of XIAP.

The term “XIAP” also includes any chimeric XIAP protein. By “chimeric XIAP” is meant a protein comprising a fusion of a XIAP domain or domains with a portion of another protein, wherein the chimeric XIAP retains the properties of human XIAP. Examples of chimeric XIAP proteins include the fusion of any of the above XIAP domains, or fragments thereof, to any domain or fragment of the following proteins such that the family has been termed Baculoviral inhibitor of apoptosis repeat-containing (Birc): Bird (NAIP1); Birc2 (cIAP1); Birc3 (cIAP2); Birc4 (XIAP); Birc5 (Survivin); Birc6 (apollon); Birc7 (livin); Birc8 (TsIAP).

The term “XIAP” is meant to include any protein with at least 70% sequence identity with human XIAP. The term also includes any conservative substitutions of amino-acid residues in XIAP. The term “conservative substitution” refers to replacement of an amino acid residue by a chemically similar residue, e.g., a hydrophobic residue for a separate hydrophobic residue, a charged residue for a separate charged residue, etc. Examples of conserved substitutions for non-polar R groups are alanine, valine, leucine, isoleucine, proline, methionine, phenylalanine, and tryptophan. Examples of substitutions for polar, but uncharged R groups are glycine, serine, threonine, cysteine, asparagine, or glutamine. Examples of substitutions for negatively charged R groups are aspartic acid or glutamic acid. Examples of substitutions for positively charged R groups are lysine, arginine, or histidine. Furthermore, the term XIAP includes conservative substitutions with non-natural amino-acids.

As used herein, the term “treating” is intended to mean the administration of a therapeutically effective amount of one of the AAV vectors described herein to a subject who is experiencing loss or impairment of hearing, loss or impairment of balance, or injury to or loss of vestibular hair cells, neurons, supporting cells, or dark cells, in order to minimize, reduce, or completely prevent or restore, the loss of hearing, the loss of balance function or of hair cells, neurons or dark cells of the vestibular portion of the inner ear. Treatment is intended to also include the possibility of inducing, causing or facilitating regeneration of the cellular elements of the inner ear including hair cells, supporting cells, dark cells, neurons and subcellular organelles of these cells including, synapses, stereocilia bundles, kinocilia, mitochondria and other cell organelles, or mechanical and functional supporting structures such as otoconia, cupula and crista of the inner ear. Treatment is also intended to prevent recurrent degeneration after regeneration of cellular elements of the inner ear, including hair cells, supporting cells, dark cells, neurons and subcellular organelles of these cells including synapses, stereocilia bundles, kinocilia, mitochondira and other cell organelles, or mechanical and functional supporting structures such as otoconia, cupula and crista of the inner ear. Treatment is also intended to mean the partial or complete restoration of hearing or balance function regardless of the cellular mechanisms involved.

As used herein, “loss of balance” or “impairment to the sense balance”, “impaired balance”, “loss of balance function” and “balance disorders” are terms that are intended to refer to a deficit in the vestibular system or vestibular function of a subject compared to the system of a normally functioning human. This deficit may completely or partially impair a subject's ability to maintain posture, spatial orientation, locomotion and any other functions associated with normal vestibular function.

As used herein, the term “administration” is intended to include, but is not limited to, the following delivery methods: topical, including topical delivery to the round window membrane of the cochlea, oral, parenteral, subcutaneous, transdermal, and transbuccal administration. In one example, the round window membrane is partially digested using a collagenase prior to transfection of the inner ear cells with an AAV vector described herein.

As used herein the term “hearing loss” is intended to mean any reduction in a subject's ability to detect sound. Hearing loss is defined as a 10 decibel (dB) standard threshold shift or greater in hearing sensitivity for two of 6 frequencies ranging from 0.5-6.0 (0.5, 1, 2, 3, 4, and 6) kHz (cited in Dobie, R. A. (2005) Audiometric Threshold Shift Definitions: Simulations and Suggestions, Ear and Hearing 26(1) 62-77). Hearing loss can also be only high frequency, and in this case would be defined as 5 dB hearing loss at two adjacent high frequencies (2-6 kHz), or 10 dB at any frequency above 2 kHz. One example of hearing loss is age-related (or aging-related) hearing loss, which is the gradual onset of hearing loss with increasing age.

As used herein, the term “prevention”, in the context of the loss of or impairments to the sense of balance, death or injury of vestibular hair cells, death or injury of vestibular neurons, injury to functionally important mechanical structures such as the ototoconia or cupula, death or injury of vestibular dark cells and the like refers to minimizing, reducing, or completely eliminating the loss or impairment of balance function or damage, death or loss of those cells through the administration of an effective amount of one of the vectors described herein, ideally before an oxidatively stressful insult, or less ideally, shortly thereafter.

Alternatively, the term “prevention” or “preventing” in the context of hearing loss is intended to refer to a significant decrease is the loss of hearing sensitivity within the aforesaid frequency range, particularly at the high frequency range 4-6 kHz.

### I. Gene Therapy

The invention features a method of treating human patients with hearing loss using full length X-linked inhibitor of apoptosis protein (XIAP), a protein that blocks apoptosis. The XIAP can be administered through gene therapy using an adeno-associated viral expression vector encoding XIAP, in which the XIAP is positioned in the vector for expression in the cells of the inner ear organ. The hearing loss is the result of inner ear organ degeneration over time, as is commonplace with aging subjects.

Generally speaking, the inner ear organ includes both the hearing and the vestibular organs (including the semicircular canals and the otolith organs (utricle and saccule). These organs have hair cells, include 1) hearing related sensory cells and supporting cells, including outer hair cells; 2) sensory cells and supporting cells and matrix and mechanical structures for sensing vestibular function (both rotation, linear motion and gravity); and 3) associated neural structures and spiral ganglion cells.

In addition to age-related hearing loss, we also contemplate that other types of hearing loss may be treatable using the expression vector described herein. Examples of other types of hearing loss include, for example: 1) ototoxicity caused by chemical or pharmaceutical agents, for example, antineoplastic agents such as cisplatinum or related compounds, aminoglycosides, antineoplastic agents, and other chemical ototoxic agents; 2) noise induced hearing loss, either from acoustic trauma or blast injury; 3) therapeutic radiation; 4) viral infections of the inner ear, such as Herpes Simplex or other viruses or infectious agents (such as Lyme Disease) that can cause inner ear hearing loss; 5) autoimmune inner ear diseases; 6) genetic hearing losses that may have an apoptotic component; 7) inner ear barotrauma such as diving or acute pressure changes; 8) physical trauma such as that caused by head injury, or surgical trauma from surgical intervention in the inner ear; and 9) inflammation or other response to administration of other inner ear regenerative compounds or gene therapy techniques.

In addition to treating hearing loss caused by inner ear cell loss using XIAP, we also contemplate that XIAP will also treat or prevent vestibular (balance) organ degeneration. Thus, XIAP gene therapy may be used to slow vestibular organ degeneration associated with aging. Vestibular loss may or may not start at the same time as hearing loss, thus XIAP may be used to simultaneously treat vestibular end organs. Vestibular organ degeneration may also result from trauma or non-trauma to the vestibular organ. Specifically, the vestibular organ degeneration may be due to ototoxicity, viral infections of the inner ear, autoimmune inner ear diseases, genetic vestibular losses, inner ear barotraumas; or physical trauma, or surgical trauma.

In general, there are two approaches to gene therapy in humans. For in vivo gene therapy, a vector encoding the gene of interest can be administered directly to the patient. Alternatively, in ex vivo gene therapy, cells are removed from the patient and treated with a vector to express the gene of interest. In the ex vivo method of gene therapy, the treated cells are then re-administered to the patient.

Numerous different methods for gene therapy are well known in the art. These methods include, but are not limited to, the use of DNA plasmid vectors as well as DNA and RNA viral vectors. In the present invention, these vectors are engineered to express XIAP when integrated into patient cells.

Adenoviruses are able to transfect a wide variety of cell types, including non-dividing cells. The invention includes the use of any one of more than 50 serotypes of adenoviruses that are known in the art, including the most commonly used serotypes for gene therapy: type 2 and type 5. In order to increase the efficacy of gene expression, and prevent the unintended spread of the virus, genetic modifications of adenoviruses have included the deletion of the E1 region, deletion of the E1 region along with deletion of either the E2 or E4 region, or deletion of the entire adenovirus genome except the cis-acting inverted terminal repeats and a packaging signal (Gardlik et al., Med Sci Monit. 11: RA110-121, 2005).

Adeno-associated virus (AAV) vectors can achieve latent infection of a broad range of cell types, exhibiting the desired characteristic of persistent expression of a therapeutic gene in a patient. The invention includes the use of any appropriate type of adeno-associated virus known in the art including, but not limited to AAV1, AAV2, AAV3, AAV4, AAV5, AAV6 and AAV7 (Lee et al., Biochem J. 387: 1-15, 2005). Previous experiments have shown that genetic modification of the AAV capsid protein can be achieved to direct infection towards a particular tissue type (Lieber, Nature Biotechnology. 21: 1011-1013, 2003). Modified serotype-2 and -8 AAV vectors in which tyrosine residues in the viral envelope have been substituted for alanine residues that cannot be phosphorylated are also contemplated. In the case of tyrosine mutant serotype-2, tyrosine 444 is substitute with alanine (t2 mut 444). In the case of serotype 8, tyrosine 733 is substituted with an alanine reside (t8 mut 733). The titer for t2 mut 444 is 4.89E+12 and that for t8 mut 733 is 7.50E+13.

The modified vectors may facilitate penetration of the vector across the round window membranes, which would allow for non-invasive delivery of the vectors to the hair cells/spiral ganglion neurons of the cochlea. The EGFR-PTK (epidermal growth factor receptor-protein tyrosine kinase) phosphorylates tyrosine residues on the surface of the capsid targeting them for ubiquitinylation and degradation by the proteosome (Zhong, L, Zhao, W, Wu, J, Li, B, Zolotukhin, S, Govindasamy, L et al. (2007) A dual role of EGFR protein tyrosine kinase signaling in ubiquitination of AAV2 capsids and viral second-strand DNA synthesis. Mol Ther 15: 1323-1330). Using t2 mut 444 or t8 mut 733 it is possible to increase gene transfer by up to 10,000 fold decreasing the amount of AAV necessary to infect the sensory hair cells of the cochlea.

Using ex vivo gene therapy, an individual skilled in the art can be assured that XIAP protein will only be expressed in the desired tissue. In these applications, as well as applications where tissue specific expression of XIAP is not a concern, the above vectors can be constructed to constitutively express XIAP protein. Numerous constitutive regulator elements are well known in the art. Often, elements present in the native viruses described above are used to constitutively express a gene of interest. Other examples of constitutive regulatory elements are the chicken.beta-actin, EF1, EGR1, elF4A1, FerH, FerL, GAPDH, GRP78, GRP94, HSP70, beta-Kin, ROSA, and ubiquitin B promoters.

For in vivo applications of gene therapy, the above vectors may be modified to include regulatory elements that confine the expression of XIAP to certain tissue types. Numerous examples of regulatory elements specific to certain tissue types are well known in the art. Of particular interest to the invention are elements that direct gene expression in the hair cells of the cochlea.

In some embodiments, it may be desirable to direct XIAP expression in an inducible fashion. Several methods of inducible transgene expression are widely used. These methods consist of the transfection of the patient's cells with multiple viral or plasmid vectors. Typically, a first vector expresses the gene of interest under the control of a regulatory element that is responsive to the expression product of a second vector. The activity of this expression product is controlled by the addition of a pharmacological compound or some other exogenous stimulation. Examples of these systems are those that respond to tetracycline, mifepristone, ponasterone A, papamycin, tamoxifen, radiation, and heat shock (Robson et al., J. Biomed. Biotechnol. 2: 110-137, 2003)

We have discovered the protective action of XIAP over-expression in the inner ear in slowing the development of presbycusis, as investigated by using a transgenic mouse in which the expression of human xiap gene is under control of the ubiquitin promoter. Our proof of concept was done using a transgene mouse model, which is engineered to produce XIAP that contains a 6-Myc tag (XIAP-Myc), and is therefore ubiquitously expressed, being present in most cells types in the cochlea. We evaluated levels of both endogenous XIAP (endo-XIAP) and the XIAP-Myc derived from the ub-xiap transgene in different tissues at distinct chronologic intervals to determine if there were any age-related changes in the levels of these proteins, and to examine any potential effects of over-expression of the ub-xiap transgene on the expression of endo-XIAP.

C57BL/6J mice are well known to express early onset (2-3 months of age) and progressive sensorineural hearing loss with ageing (Mikaelian, 1979; Henry and Chole, 1980; Willott, 1986; Hunter and Willott, 1987; Li and Borg, 1991; Spongr et al., 1997). Moreover, the aging process of this species appears to be more rapid in the inner ear than in the brain, resulting in “old ears” connected to a young brain at the relatively early-to-middle life span of the animals (Willott, 1986; Parham and Willott, 1988). The reason(s) for this faster aging specifically in the auditory system remains to be explored. Up to three major genes have been identified as major contributors to AHL in mice, and each mouse strain examined may contain one to three of these genes (Erway et al., 1993). For example, a major AHL gene has been mapped in C57BL/6J mice at chromosome 10 and the same gene has been found to be a major contributor to AHL in nine other inbred mouse strains (Erway et al., 1993; Johnson et al., 1997; Johnson et al., 2000). However, it is not known how many different AHL genes are present collectively in each mouse strain and the differences in AHL onset and development across different species cannot be attributed to the allelic heterogeneity of the AHL genes (Johnson et al., 2000). Further, it is not known how these genes are related to the apoptotic cell death seen during AHL.

Previous studies have suggested that apoptosis is involved in degenerative cell death in brain as well as in aged cochleae (Zheng et al., 1998; Alam et al., 2001; Iwai et al., 2001; Spicer and Schulte, 2002; Pickles, 2004). A significant increase of caspase-3 with aging was reported in the organ of Corti, in SGNs, and in the lateral wall of the cochlea in gerbils (Zheng et al., 1998; Alam et al., 2001). The apoptosis pathway can be triggered by various mechanisms in the cochlea of aging gerbils, including accumulated damage from free-radicals and deteriorating mitochondrial function and structure (Zheng et al., 1998). Very recently, induction of apoptotic markers has been correlated with mutations in mitochondrial DNA (mtDNA) accumulated during aging, and with increased markers of oxidative stress (Kujoth et al., 2005). Accumulated mtDNA mutation has long been associated with presbycusis (Seidman et al., 2002; Ohlemiller, 2004). The correlation between the accumulation of mtDNA mutation and apoptotic markers suggests that the mtDNA mutation may promote apoptosis as a cause of cell death during aging (Kujoth et al., 2005). Although AHL is defined as a hearing loss due to aging without significant insults from hazardous factors, it is possible that cochlea presbycusis occurs as the consequence of interplay between hazardous environmental events and genes that govern protection and repair of the cochlea cells (Johnson et al., 2000). For example, several studies have showed that the AHL gene renders C57 mice more susceptible to noise induced hearing loss (NIHL). Nonetheless, it is clear that apoptosis plays an important role for the cochlea lesions accompanying the development of AHL.

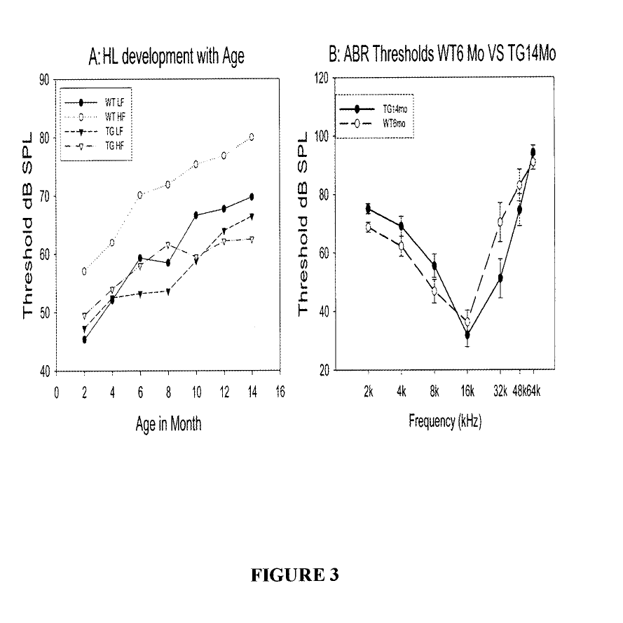

We have discovered that the aging-related hearing loss (AHL) in the C57BL/6J mouse strain can be significantly delayed by the over-expression of XIAP. This protection is particularly apparent at the high-frequency region in which hearing sensitivity in 14 month old TG mice is approximately the same as 6 month WT mice (FIG. 3B). The cytoprotective effect of XIAP is also clearly demonstrated by the significantly less hair cell loss in TG relative to WT mice evaluated at 14 months of age. This result suggests that the cochlea hair cells are dying, at least in part, during the process of aging through apoptosis.

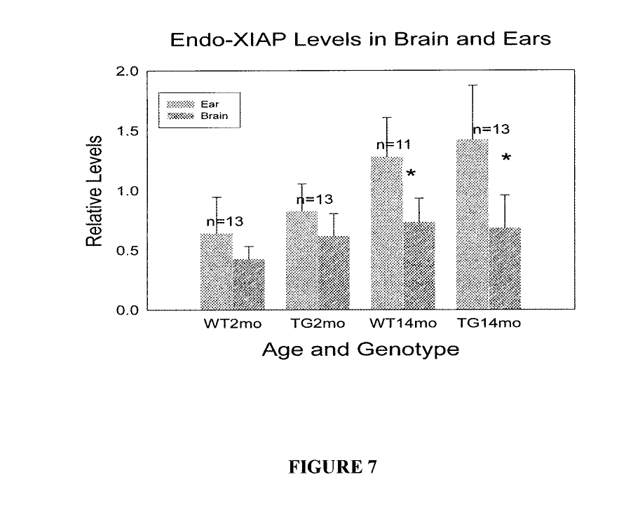

We have also discovered a unique age-related increase in endo-XIAP in the cochlea. This result suggests that the endo-XIAP accumulates in the cochlea in response to the activation of apoptosis with aging. Interestingly, the increasing endo-XIAP with aging was not seen in brain tissue from the same mice, suggesting that apoptosis is more predominant and severe in the cochlea than in the brain. This finding is consistent with the fact that the aging in the cochlea is quicker than in the brain in this strain and provides additional support to the role of apoptosis in AHL. XIAP is thought to be ubiquitously expressed and is translated to produce anti-apoptotic properties in response to a variety of apoptosis-inducing conditions. In contrast, other IAPs show either a more limited expression pattern or inhibit a relatively limited subset of apoptotic triggers. Therefore, a housekeeping function for XIAP may exist. Nevertheless, the level of XIAP in healthy tissue is generally low, as shown in the brain and ears of younger age mice in this experiment (FIG. 7), with an increase in the level of XIAP in response to different stressors. Inferring from the early onset of hearing loss, it appears that apoptosis is triggered more easily in the cochlea than in the brain in this strain, at this stage for known reasons.

We have further discovered that the level of XIAP-Myc, which arises from the transferred human xiap gene, appears to remain unchanged with age. This indicates that transgene may not be regulated in response to the apoptosis accompanying AHL. Rather, a stabilized expression is provided by the transgene. Without wishing to be bound by theory, we believe that it is this age-stabilized XIAP that provides the extra protection against apoptosis in the transgenic mice; and this stabilized expression does not suppress the expression of endogenous xiap gene, based on the fact that the endo-XIAP levels in the cochlea appears to be similar in the two genotype groups. It also appears that the endo-XIAP, despite its increase with aging, is not adequate to fully protect the cochlea from apoptotic cell death. The extra quantity of XIAP provided by the transgene seems to help to protect the cochlea from aging.

Although great efforts have been made to understand the biological control of XIAP expression, it is still not entirely clear how XIAP expression is regulated in responses to various insults during apoptosis. XIAP has been found to be under transcriptional control by the stress-inducible transcriptional activator NF-κB (Stehlik et al., 1998) and to be regulated at the level of protein synthesis by ubiquitination (post-translation regulating). However, it is also possible that transcription of the gene is promiscuous (Holcik, 2003), and that regulation is mostly post-transcriptional to allow for differential expression in tissues that require more or less XIAP protein. This possibility is supported by the extensive 5- and 3-untranslated regions (UTRs) in the messenger RNA of XIAP and the internal ribosome entry site (IRES) in the 5-UTR (Holcik et al., 1999). Several binding proteins have been described which regulate XIAP expression at the RNA level (see review by (Holcik, 2003)). It has been suggested that the IRES-mediated translation allows for enhanced expression of XIAP when cap-dependent translation protein synthesis is shut off or compromised following the induction of apoptosis (Hellen and Sarnow, 2001). Increased expression of endo-XIAP has been reported in various conditions of cellular stress (Holcik et al., 1999; Holcik et al., 2000a; Holcik et al., 2000b). XIAP activity has also been reported to be under the control of two negative regulators, termed XIAP associated factor 1 (XAF1) (Fong et al., 2000; Liston et al., 2001) and direct IAP binding protein with low pl (Smac/DIABLO) (Du et al., 2000; Verhagen et al., 2000) at post-translation level.

Because only the coding domains of the human xiap gene are transferred in the XIAP-Myc mice, it is possible that the finding of stabilized XIAP-Myc with aging is due to the lack of regulating sites in the mRNA from the transgene. The stabilized expression of XIAP-Myc with aging also suggests that this expression is not under the control of the two negative regulators mentioned above. In a recent study using viral vector for xiap gene transfection, it was suggested that the exogenous XIAP exerted its hair cell protection against cisplatin ototoxicity mainly by combining with one of the two negative regulators, namely Smac/DIABLO, based on the finding that the transgene modified to not contain the binding site for Smac/DIABLO did not provide this protection (Chan et al., 2007). This study suggests that the binding of exogenous XIAP with Smac/DIABLO frees the endo-XIAP to counteract apoptosis. However, this is unlikely to be the mechanism of protection provided by the XIAP-Myc in the aging cochleae in our experiments. Since there is no mechanism for the up-regulation of XIAP-Myc, binding with the negative regulator would likely exhaust this exogenous XIAP. However, we did not see an age-related decrease in XIAP-Myc level. The increase of endo-XIAP with age is likely due to the positive regulation through internal ribosome entry site (IRES) in the 5-UTR that occurs at a post-transcriptional level. Nevertheless, the total level of XIAP is increased with the addition of XIAP-Myc in the cochleae of in the TG group. This increase appears to be effective in delaying aging in the cochlea in this species.

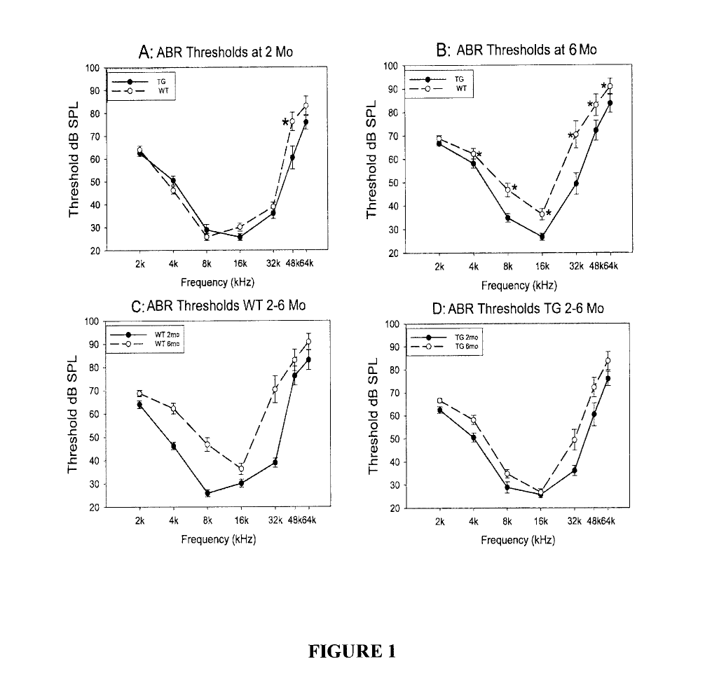

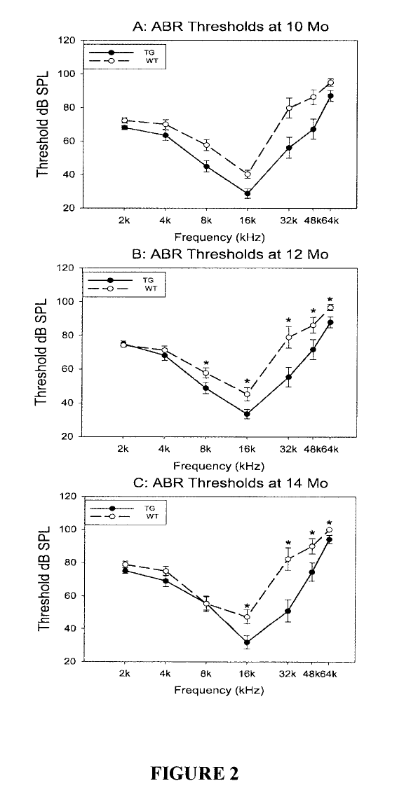

We have also discovered that XIAP-Myc provides better protection at the high-frequency region than in the low frequency region. Consistent with previous reports (Mikaelian, 1979; Li and Borg, 1991; McFadden et al., 2001), our ABR results show that the age-related hearing loss starts at the high-frequency end of the hearing range of the mice, and spreads out towards middle and low frequency regions with ageing. A significant hearing loss in the low-to-middle frequency regions has also been reported in the previous studies and is confirmed in the present study. To our surprise, we found that the LF hearing loss is not a result of a downward spreading from the high-frequency regions, but rather is separately initiated in the low-frequency regions. This LF hearing loss was evident even when comparing the hearing of 2 and 4 month old mice, especially in WT groups (FIG. 1). The hearing loss in this region developed slightly later and progressed much slower in the TG group than in the WT group up to 8 months of age (FIG. 3A). But the LF loss was significant by the end of the experiment in both genotype groups. The LF hearing loss appears to accelerate after 8 months of age in TG groups so that there is no statistical difference between the two groups (FIGS. 2 and 3). More importantly, the amount of hair cell loss appears to match the degree of hearing loss in the high frequency region in the two groups. In the low frequency region, however, we generally see no hair cell loss in the TG group and only a slight hair cell loss in the WT group. Therefore, the LF hearing loss is not due to hair cell loss. A similar discrepancy between the hair cell loss and the elevation of the thresholds has also been demonstrated in previous studies (Spongr et al., 1997; McFadden et al., 2001). Since we did not observe pathology in other part of the cochleae, we do not know which degenerative changes are responsible for the LF hearing loss shown in this species during aging. Since the LF hearing loss is not protected by XIAP over-expression, the pathology or degenerative changes involved may not be due to apoptosis.

In the predominant conceptual framework for AHL or presbycusis proposed by Schuknecht (Schuknecht, 1964; Schuknecht and Gacek, 1993), the three major cochlea elements (organ of Corti, SGNs, and Stria vascularis (SV)) can degenerate separately, thereby contributing to AHL independently. The apical turn of the cochlea has been found to be prone to primary SGN loss in humans and many animal species including C57 mice (Covell and Rogers, 1957; Keithley et al., 1992; Felder and Schrott-Fischer, 1995; Dazert et al., 1996; Willott et al., 1998; Ohlemiller and Gagnon, 2004b). In some recent reports, pathology related to the SGN loss have been reported to show apical-to-basal gradient during the development of presbycusis (Ohlemiller, 2004; Ohlemiller and Gagnon, 2004a, 2004b). This is opposite to the HC loss that is develops from the basal turn to the apex. These include the abnormalities of the spiral limbus, pillar cells and Reissner's membrane. However, it is not clear how these changes are related to the death of SGNs and if these changes are due to apoptosis.

Even though we were unable to examine the anatomy of the SV and SGNs, we think that a postulated non-apoptotic lesion is more likely to occur in the strial vascularis (SV), but not in the SGNs. This is supported by the previous studies which suggest that apoptosis is always involved in aging related SGN death, while the degenerative changes in the SV can be atrophic in nature. In one report, for example, DNA fragmentation (an indicator of an endonuclease activation seen in apoptosis) was found predominantly in the OHCs and SGNs, but not in the stria cells, which showed a marked atrophy (Zheng et al., 1998). A later study reported that the expression of a particular protein in the apoptotic pathway (the caspase-3p20) increased with ageing in the organ of Corti, SGNs, as well as lateral wall of the cochlea in gerbils. This increase in active caspase was claimed to be compatible with functional hearing deterioration (Alam et al., 2001). To our knowledge, data about the topographic quantification of degenerative changes in the SV is not available in the C57 species. In one study, SV atrophy was presented at the basal turn but not at the apical low-frequency region where the discrepancy occurred (Mikaelian, 1979).

Another possible explanation for the LF hearing loss that does not involve the HC is conductive hearing loss from middle ear pathology, which is often low-frequencies biased. Although we did not see any obvious middle ear abnormalities or fluid by visual inspection when cochleae were harvested at the end of the experiments, this simple inspection cannot rule out entirely the possibility of ageing related changes in the middle ear structures.

We have therefore demonstrated that over-expression of XIAP by genetic manipulation provides protection in C57 mice against age-related hearing loss, and this loss is probably is a result of the accumulation of apoptotic processes in the cochlea with aging. The transferred XIAP gene is not regulated in response to apoptosis, rather, it provides a steady baseline activity throughout the mouse's life span. The transferred gene does not interfere with the expression of the endo-XIAP gene. The endo-XIAP gene expression increases with ageing in the cochlea but not in the brain of C57 mice, suggesting that the cochlea is the more predominant site for apoptosis in this species. Over-expression of XIAP provides protection against AHL in the high-frequency region but not in the low frequency region where the degenerative pathology may not be apoptotic, or inner ear related.

Our evidence suggest that apoptosis plays a significant role in age-related hearing loss (AHL) or presbycusis. We evaluated whether over-expression of the anti-apoptotic protein known as X-linked Inhibitor of Apoptosis protein (XIAP) slows the development of presbycusis. We compared the effect of aging on the hearing status of transgenic (TG) mice that over-express human xiap under control of the ubiquitin promoter on a pure C57BI/6 genetic background with wild-type (WT) littermates. In order to distinguish endogenous XIAP from that derived from the transgene, the transgenic XIAP was engineered to contain a 6-Myc tag (XIAP-Myc). Auditory brainstem responses (ABR) were measured every two months from 2 to 14 months of age. Hair cell loss in the cochlea was assessed by cochleograms following the final ABR testing. Comparison of the levels of endogenous XIAP (endo-XIAP) and XIAP-Myc over time demonstrated that the transgene elevated total XIAP by up to 50% in the cochlea and by 100% in the brain, and that XIAP-Myc level did not change with aging in either tissue. In contrast, endo-XIAP appears to increase with ageing in the cochlea, but not in the brain. ABR measurements showed that WT mice developed hearing loss much faster than XIAP-Myc mice. XIAP over-expression reduced hearing loss associated with aging, particularly within the high-frequency range. The average total hair cell loss per-cochlea was 665.47±417.99 (mean±SD) in the WT group compared to 219.95±258.37 in the TG group (t-test, t=−4.221, p<0.001). Taken together, these results suggest that XIAP over-expression reduces age-related hearing loss and hair cell death in the cochlea. Treatment strategies based on elevation of XIAP may, therefore, have utility in the treatment of presbycusis.

### II. Pharmaceutical Compositions and Modes of Administration

The vector used with some embodiments as described herein can be incorporated into pharmaceutical compositions suitable for administration to a subject. In some particular embodiments, the pharmaceutical composition comprises the vector of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it can be preferable to include isotonic agents, for example, sugars, polyalcohols such as mannitol, sorbitol, or sodium chloride in the composition. Pharmaceutically acceptable carriers can further comprise minor amounts of auxiliary substances such as wetting or emulsifying agents, preservatives or buffers, which enhance the shelf life or effectiveness of the vector or pharmaceutical composition.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form used depends on the intended mode of administration and therapeutic application. Typical compositions are in the form of injectable or infusible solutions, such as compositions similar to those used for passive immunization of humans. The typical mode of administration is intratympanic (in the middle ear), intracochlear, parenteral (e.g., intravenous, subcutaneous, intraperitoneal, intramuscular, intrathecal). In one example, the vector is administered by intravenous infusion or injection. In another example, the vector is administered by intramuscular or subcutaneous injection. In another example, the vector is administered perorally. In yet another example, the vector is delivered to a specific location using stereostatic delivery, particularly through the tympanic membrane or mastoid into the middle ear.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the vector in the required amount in an appropriate solvent with one or a combination of ingredients enumerated above, as required, followed by filtered sterilization.

Generally, dispersions are prepared by incorporating the vector into a sterile vehicle that contains a basic dispersion medium and the required other ingredients from those enumerated above. In the case of sterile lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be achieved by including an agent in the composition that delays absorption, for example, monostearate salts and gelatin.

The vector of the present invention can be administered by a variety of methods known in the art. As will be appreciated by the skilled artisan, the route and/or mode of administration will vary depending upon the desired results. In certain embodiments, the vector may be prepared with a carrier that will protect the vector against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are generally known to those skilled in the art.

The pharmaceutical compositions of the invention can include a “therapeutically effective amount” or a “prophylactically effective amount” of the vectors of the invention. A “therapeutically effective amount” refers to an amount effective, at dosages and for periods of time necessary, to achieve the desired therapeutic result, in this case for both prophylaxis and treatment of hearing loss or impairment of balance without unacceptable toxicity or undesirable side effects.

A therapeutically effective amount of the vector can vary according to factors such as the disease state, age, sex, and weight of the individual, and the ability of the vector to elicit a desired response in the individual. A therapeutically effective amount can also be one in which any toxic or detrimental effects of the vector are outweighed by the therapeutically beneficial effects. A “prophylactically effective amount” refers to an amount effective, at dosages and for periods of time necessary, to achieve the desired prophylactic result. Typically, since a prophylactic dose can be used in subjects prior to or at an earlier stage of disease, the prophylactically effective amount can be less than the therapeutically effective amount.

Dosage regimens can be adjusted to provide the optimum desired response (e.g., a therapeutic or prophylactic response). For example, a single bolus can be administered, several divided doses can be administered over time or the dose can be proportionally reduced or increased as indicated by the exigencies of the therapeutic situation. It can be especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of vector calculated to produce the desired therapeutic effect in association with the required pharmaceutical carrier. The specification for the dosage unit forms of the invention can be dictated by and directly dependent on (a) the unique characteristics of the vector and the particular therapeutic or prophylactic effect to be achieved, and (b) the limitations inherent in the art of formulating such vector for treating or preventing hearing loss or impaired balance in a subject.

It is known that in the auditory system, three major viral vectors have been investigated for cochlear gene transfection: (1) lentivirus, (2) adenovirus and (3) Adeno-associated virus (AAV). The gene transfected by adenovirus vector has limited expression time and the vector has been associated with adverse immune reactions (Staecker, Brough, Praetorius, & Baker, 2004). The lentivirus vector, although capable of maintaining long term expression, is particularly suited for targeting neurons, but not hair cells (Federico, 1999). Since the AAV vector has several advantages such as long lasting expression of synthesized genes (Cooper et al, 2006), and low risk for pathogenic reactions (because they are artificially manufactured and not ototoxic) (Kaplitt et al., 1994), it is likely to be the best choice of viral vector for cochlear protection by gene therapy.

Cochlear gene transfection in animals has utilized several approaches for vector delivery: (1) direct injection through round window membrane (RWM) into the perilymph, (2) intracochlear infusion through cochleostomy, and (3) transfusion through an intact RWM (Aarnisalo, Aarnisalo, Pietola, Wahlfors, & Jero, 2006). The third approach (transfusion through intact RWM) is least invasive and most likely to be accepted in human application. Until now, it was known that RWM is not permeable to AAV (Jero et al, 2001).

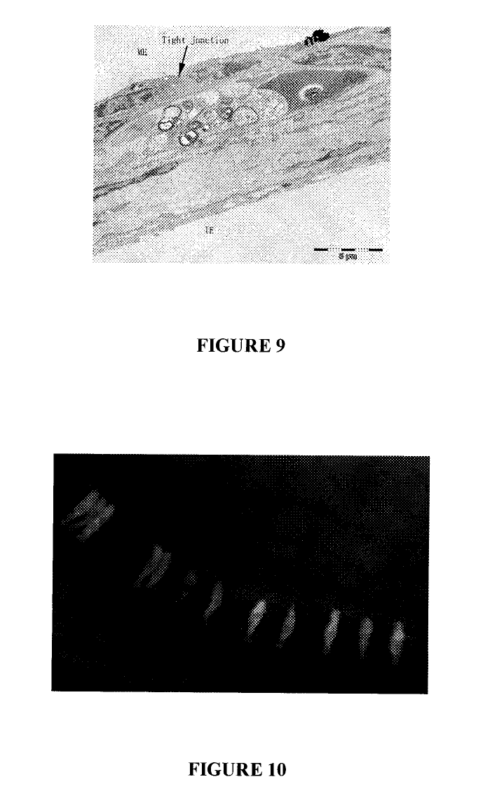

The intact RWM consists of three layers: two epithelia layers separated by a layer of connective tissue (FIG. 9), with collagen being a major component of the RWM. We have now demonstrated that the permeability of RWM can be increased temporarily by digestion of the membrane with collagenase. We then investigated whether (1) the digestion of RWM with the enzyme could facilitate the gene transfection of inner ear cells, and (2) if the digestion was safe to inner ear function and the structure of RWM.

### Methods:

### 1: Transgenic Mouse and Expression Vector Production

Transgenic founders were generated by microinjection of a linearized plasmid construct consisting of the Ubiquitin C promoter, 6 repeats of the 9E10 myc epitope tag fused to the amino terminus of the human XIAP coding region, and a polyadenylation signal from SV40. The construct was microinjected into the male pronucleus of C57BI/6×C3H Fl zygotes. All lines were maintained in the heterozygous state by cross breeding with wild type C57BI/6 mice. Transgene status within the colony was determined by PCR targeting 6-myc tag.

The XIAP-Myc C57 transgenic (TG) mice and wild-type (WT) littermates were bred in the animal facility at Dalhousie University. In total, 48 mice were recruited into this study for longitudinal observation of the development of hearing loss with time. There were 24 in each of the WT and TG groups with matched number of mice of each gender in the two groups. During the 14 months of observation, some mice died for various reasons. At the end of the experiment, 17 TG and 15 WT mice survived. Hearing status was evaluated using frequency-specific auditory brainstem responses (ABR) that were performed every two months from the ages of 2 months to 14 months. After the final ABR testing, the animals were sacrificed and the both cochleae were harvested; one was used for evaluation of hair cell loss and the other for the quantification of XIAPs. In total, 19 cochleae were taken from each group for cytocochleograms. Western blotting was employed for the quantification of both XIAP-Myc and endogenous XIAP. A piece of brain tissue was also taken from each animal for XIAP testing as well. Western blotting was successful in 14 cochleae in the TG group and 11 in the WT group. Correspondingly, Western blotting was performed using brain tissue from the same mice in each group. To investigate in more detail the effects of aging on XIAP expression, an additional 34 young mice (2 months old, 17 in each genotype group) were recruited to evaluate XIAP expression with Western blotting.

For administration of a vector to treat age-related hearing loss, the adeno-associated virus serotype 2 (AAV-2) construct is used. The adeno-associated virus serotype 2 (AAV-2) construct includes a myosin7a promoter to drive expression of XIAP in the outer hair cells that are necessary for high frequency hearing and lost with aging. It should be noted that outer hair cell loss and inner hair cell loss can be targeted using the vector. Moreover, the expression promoter may be ubiquitin. This is done in two ways 1) by direct injection of the AAV2-XIAP using saline as a vehicle, as a composition, into the cochlea or by simply injecting this construct past the tympanic membrane so that it will be taken up by the round window membrane of the cochlea in sufficient amounts to infect the outer hair cells. In other methods, the agent may be applied onto an absorbable material such as Gelfoam® that is placed against the round window, and delivers the vector to the round window. In other methods of delivery, an active controlled release pump is used to direct the agent in solution at a predefined rate to the round window area. In other methods of delivery, a passive wick is placed against the round window membrane, and the agent is applied to the lateral end of this wick for delivery by wicking action to the round window membrane. Direct routes to the cochlea may include a fenestra into the stapes footplate, round window membrane, labyrinth (semicircular canals), promontory, or via the internal auditory canal through CSF and neural pathways.

AAV Vector preparations are produced by the plasmid cotransfection method [S. Zolotukhin et. al. Gene therapy 1999]. Briefly, one cell factory (Nalgene Nunc International, Rochester, N.Y., USA) with approximately 1×10⁹ HEK 293 cells is cultured in Dulbecco's Modified Eagle's Medium supplemented with 5% fetal bovine serum and antibiotics (cDMEM). A CaPO4 transfection precipitation is set up by mixing a 1:1 molar ratio of rAAV vector plasmid DNA and serotype specific rep-cap helper plasmid DNA. This precipitate is added to 1100 mL of cDMEM and the mixture is applied to the cell monolayer. The transfection is allowed to incubate at 37° C. for 60 h. The cells are then harvested and lysed by three freeze/thaw cycles. The crude lysate is clarified by centrifugation and the resulting vector-containing supernatant is divided among four discontinuous iodixanol step gradients. The gradients are centrifuged at 350,000 g for 1 h, and 5 ml of the 60-40% step interface is removed from each gradient and combined.

This iodixanol fraction is further purified and concentrated by column chromatography on a 5-ml HiTrap Q Sepharose column using a Pharmacia AKTA FPLC system (Amersham Biosciences, Piscataway, N.J., USA). The vector is eluted from the column using 215 mM NaCl, pH 8.0, and the rAAV peak collected. Vector-containing fractions are then concentrated and buffer exchanged in Alcon BSS with 0.014% Tween 20, using a Biomax 100K concentrator (Millipore, Billerica, Mass., USA). Vector is then titered for DNase-resistant vector genomes by Real-Time PCR relative to a standard. Finally, the purity of the vector is validated by silver-stained SDS-PAGE (the three AAV capsid proteins are the only visible protein bands in an acceptable prep), assayed for sterility and lack of endotoxin, and then aliquoted and stored at −80° C.

### 2: ABR Measurement

The mouse was anesthetized with a ketamine and Xylacine mixture (60-80 mg/kg+10 mg/kg respectively i.p.) and put on a thermostatic heating pad to keep the body temperature at 38.5° C. Signal generation and ABR acquisition employed Tucker-Davis hardware and BioSig software (Tucker-DavisTechnology system III). The stimuli consisted of tone bursts at 2, 4, 8, 16, 32, 48 and 64 kHz, with a duration of 10 ms and rise/fall of 1 ms (Blackman window). The stimulation rate was of 21.1/sec, and 1000 evoked responses were averaged for each trial. At each frequency, the ABR was tested by starting with 90 dB sound pressure level (SPL) and then decreasing stimulation SPL in 5-10 dB steps until the threshold for detecting a repeatable response was reached. The evoked responses were recorded by sub-dermal electrodes, band-pass filtered between 100-3000 Hz, before amplification. If the evoked response was not detected at the highest sound presentation level (90 dB SPL) at any given frequency, the threshold at this frequency was labeled as 100 dB SPL

### 3: Cytocochleogram

The methods for determining cochlea morphology were similar to those reported by others in the past (Ding et al., 1999b; Ding et al., 2001). The cytocochleogram was determined by the spatial-percentage count of missing hair cells along the cochlea duct. To do this, the mouse was deeply anesthetized with an over-dose of Ketamine, and the cochlea rapidly harvested after the final ABR test. Surrounding soft tissues were removed, and the round window and oval window were both opened. A small hole was made with a needle at the apex of the cochlea for perfusion and staining. The staining solution for succinate dehydrogenase (SDH) histochemistry was freshly prepared by mixing 0.2 M sodium succinate (2.5 ml), phosphate buffered saline (2.5 ml) and nitro-tetranitro blue tetrazolium (nitro-BT, 5 ml). The cochlea was gently perfused through the hole at the cochlea apex and the opened round and oval windows. Following this, the cochlea was immersed in the SDH solution for 45 min at 37° C., and then fixed with 10% formalin for 4 hours. After fixation, the cochlea was decalcified with 5% EDTA solution for 72 hours. The organ of Corti was dissected and surface preparations were made on slides. Cytocochleograms were established using normative data for C57 mice with custom-made software.

### 4: Western Blotting

Western blotting was employed to quantify the both endogenous XIAP and XIAP-Myc in both cochleae and brain tissues. Soft tissue was harvested as much as possible from each cochlea, and a 2 mm3 piece of brain tissue was also taken from the temporal lobe of each mouse. Tissues were homogenized in RIPA buffer (1% Triton X-100, 1% SDS, 8.77% NaCl, 2.42 Tris-HCl base and 5% Deoxycholic acid, pH 8) and then centrifuged at 14,000 g for 10 min at 4° C. Supernatants were transferred to a new 1.5 ml tube. Protein concentrations were estimated using Bio-Rad reagent and a microplate reader (ELx 800 UV, Bio-tek Instrument Inc.). Following this, 20 μg of protein from each sample was transferred into a tube containing RIPA, 2×SDS sample buffer (7.5 pt each) and DTT (15 mg/mL). The sample was stored at −80° C. for later use. The sample was then separated by 10-15% SDS-polyacrylamide gel electrophoresis in running buffer and then transferred to PDVF membrane. The membrane was blocked in blocking solution (containing 1M Tris-HCl 25 ml, 1M NaCl 150 ml and Tween-20 500 μl, 5% non-fat milk powder in 1 Liter) overnight at 4° C. The blots were then probed with a primary antibody directed against the epitope of both endogenous the XIAP and the XIAP-Myc (1:1,500, XIAP Ab mouse, BD Biosciences 610762), in addition to an antibody for β-actin (1:20,000; Sigma A5441), followed by anti-mouse IgG horseradish peroxidase-linked antibody (1:10,000; Vector Laboratories, PI-2000). Band detection was achieved using ECL Plus Kit (GE Health Care) and read with the Storm 840 gel analysis system. The β-actin band was used as internal reference for the level of both endo-XIAP and XIAP-Myc.

### 5: Data Analysis

Hearing was documented in audiograms, which show the ABR thresholds as a function of frequency, and compared between the two groups over time. Two-way ANOVA was performed against the factors of genotype and frequency at each time point when hearing sensitivity was evaluated with ABR. If a significant effect of genotype factor was found, post-Hoc tests will be done to verify at which frequencies, the difference between the two groups would be significant. Hair cell loss was documented in the cochleograms of the surviving 17 mice in the TG group and the 15 mice in the WT group. Loss of both inner hair cells (IHCs) and outer hair cells (OHCs) was counted, and the total loss was compared between the two groups.

### 6: Injection Methods

For animal modeling, the following techniques can be used.

A. Round window method

Adult guinea pigs or rats are anesthetized. A dorsal postauricular incision is made and the bone medial to the tympanic ring is exposed. A hole is drilled exposing the middle ear space medial to the tympanic ring. The round window niche and the bone overhanging the niche are both identified. The bone is scraped away revealing the round window membrane. Vector injections are carried out using a microsyringe. A needle is used to puncture the round window membrane using a micromanipulator while the animals are immobilized to minimize injection trauma. After the injection, the round window is patched with a small piece of muscle tissue. For each approach, control animals are injected with artificial perilymph to control for damage induced by hydraulic forces. Hearing is tested by ABR audiometry.

For the round window route, it is also possible to use transtympanic injection, and to allow either active or passive uptake of the vector via the round window membrane without puncture. In other methods, the agent may be applied onto an absorbable material such as Gelfoam® that is placed against the round window, and delivers the vector to the round window. In other methods of delivery, an active controlled release pump is used to direct the agent in solution at a predefined rate to the round window area. In other methods of delivery, a passive wick is placed against the round window membrane, and the agent is applied to the lateral end of this wick for delivery by wicking action to the round window membrane.

### B. Semicircular Canal Method

After surgically exposing the temporal bone, the superior semicircular canal is identified. An argon, KTP, CO₂ or other laser is used to create an opening in the bone. Injection of the vector and data acquisition is carried out.

### C. Cochleostomy

Animals are prepared as previously described above. The promontory overlying the basal turn of the cochlea was identified. An argon, KTP, CO₂ or other laser is used to create a cochleostomy anterior to the round window, or inferior to the oval window. Injection of vector is then carried out. The cochleostomy is sealed and the animal is allowed to recover.

Additionally, the vector can be injected into the cerebro-spinal fluid (CSF) and monitor the vector's progress via cochlear aqueduct or internal auditory canal into inner ear.

### 7: Vector Delivery and Transfection in Humans

For human subjects, injection may be done via the lateral semicircular canal, which can be accessed either by drilling or by use of a laser, such as for example, a CO₂ laser. In one example, a saline solution of the vector which encodes for X-linked inhibitory protein (XIAP) is injected into the inner ear via a hole prepared as above. Transfection of XIAP causes diminution of the apoptosis and loss of hearing associated with aging and can allow surgical intervention days after the initial transfection, as the transfected hair cells will be producing XIAP. Additionally, the vector can be administered to a human subject by a stapedotomy or via diffusion of the vector across the round window membrane, including placing the vector onto an absorbable carrier such as gel foam, or non-absorbable carrier in contact with the round window membrane or via an active micro-pump or passive wicking system to the round window.

The level of XIAP (both endo-XIAP and XIAP-Myc) was calculated as a volume ratio between the XIAP and β-actin. The expression of endogenous XIAP was evaluated against three factors (age: 2 verses 14 months, genotype: TG versus WT, and tissue: cochlea versus brain) in 3 way ANOVA using α=0.05 as significance level. Post Hoc paired tests were performed within the age and tissue factors, because these factors achieved significance on ANOVA testing. The expression of XIAP-Myc was also evaluated against age and tissue type in two-way ANOVA (p<0.05).

8. Enzyme Facilitated Cochlear Gene Transfection with AAV

Under appropriate anesthesia, the round window of the guinea pig was surgically exposed and inspected visually under a surgical microscope. Freshly prepared collagenase solution of 2-3 ul was applied to the round window niche with the help of a microinjection pump. The reaction time was 10 minutes. The residual solution was then sucked out and the RWM was washed with saline. The collagenase was used at a concentration of between 50 unit/ml to 200 unit/ml. In one example, the concentration used was 150 unit/ml. In the example provided here, the collagenase was collagenase from Clostridium histolyticum, Type II (available from Sigma). Other proteolytic enzymes such as, for example, papain, trypsin, pepsin, chymotrypsin or elastase could also be used to partially digest the round window membrane. Also, a miniosmotic pump can be used to continuously deliver the AAV in the vicinity of the round window membrane so as to saturate the membrane with the AAV vector.

To evaluate the damage of the digesting solution on RWW, the animal was killed immediately after the digestion and the cochleae were taken out and fixed with standard protocol for electronic microscopy. To evaluate if the damaged RWM heals spontaneously, some other animals were allowed to survive for 3 weeks before the RWMs were taken for examination.

For inner ear gene transfection, a piece of gelfoam (1-2 mm³) was placed gently on RWM. 5 ul AAV solution was applied to the gelfoam. The cochlea was taken out for cochlea immunostaining against GFP 2-4 weeks after the surgical application of AAV vectors.

ABR was tested in animals underwent gene transfection before and just before the animal was killed for morphology.

This procedure is also applicable in the use of the adeno-associated viral expression vectors, or mutants thereof, which encode XIAP so as to locally transfect the cells.

### 9. Surgical Procedure for AAV Injection

Patients identified as suffering from aging related hearing loss would be eligible for XIAP gene therapy. The cochlea would be accessed via the round window membrane (RWM) that has been partially permeabilized using a proteolytic enzyme such as collagenase. The RWM in turn would be accessed via a small incision in the tympanic membrane. Specifically, a myringotomy (surgical procedure in which a small incision is created in the eardrum) will be performed to access the round window membrane (RWM) under local anesthetic as an out patient procedure in adults. Collagenase will be applied to the round window membrane (10 ul) at a concentration of 50 unit/mL for 10 min. After this time, the collagenase solution in contact with the round window membrane is aspirated and the membrane washed with sterile solution to remove any residual enzyme. Our analysis of the round window membrane by transmission electron microscopy has shown that the damage produced by collagenase treatment is temporary and by 3 weeks the round window membrane (RWM) has nearly completely healed. 20 ul AAV solution (tyrosine mutant AAV serotypes 2-8, titer 1-5E13) will be applied to gelfoam. For transfection of sensory hair cells and related conduction cells, the AAV containing gelfoam (5 mm3) will be placed gently on the RWM. The gelfoam will be allowed to remain in contact with the RWM where it is slowly broken down and absorbed into the surrounding tissue in a non-injurious manner. This procedure is also applicable in the use of the adeno-associated viral expression vectors, or mutants thereof, which encode XIAP so as to locally transfect the cells. Other methods of delivery include active micropumps, and passive wicking systems.

### Results

I. Hearing Loss Progress with Age

In general, C57BU6 mice rapidly develop hearing loss starting at very early age (e.g., 2 months). In comparison to WT littermates, TG mice displayed better ABR thresholds at 2 months of age. At this age, hearing loss predominately affected the high frequency regions (FIG. 1A). At this time, the differences in averaged thresholds were found to be larger than 5 dB only at the two highest frequencies (48 and 64 kHz) tested. The thresholds were 76.19 and 83.04 dB SPL at 48 and 64 kHz for WT mice, 60.41 and 75.93 dB SPL for TG mice at the two frequencies. Two-way ANOVA indeed identified a significant effect of genotype. The difference between the groups was found to be significant at 48 kHz (Mann-Whitney Rank Sum Test, p<0.05, as indicated by the asterisk in FIG. 1A) but not at 64 kHz. The development of hearing loss was found to be slower in the TG group. This is demonstrated in two different ways in FIG. 1. FIG. 1B shows the ABR audiograms at the age of six months. Significant difference was found in favor of TG group at frequencies of 8 kHz and above, suggesting a slower development of high-frequency hearing loss in the TG group. FIGS. 1C and D show the changes of ABR thresholds from 2 months to 6 months in the two groups. In the TG group, the ABR thresholds generally remain unchanged at frequencies below 16 kHz from the values at 2 months of age (FIG. 1D). In the WT group, however, the threshold elevation was found to be larger than 5 dB at all frequencies and the change was statistically significant at the frequencies indicated by asterisks (FIG. 10).

In the later stages of the experiment, the threshold differences between the two groups were further intensified in the high frequency regions, but the opposite was seen in the low frequency regions. FIG. 2 shows the ABR threshold changes observed at 10, 12 and 14 months. At 10 months, TG mice show superior thresholds across all the frequencies tested. Comparison of the data in A, B and C reveal that TG animals retain superior ABR thresholds in the high-frequency region (16, 32, 48 and 64 kHz) relative to WT mice; however, within the low frequency region (2, 4 and 8 kHz) the differences become smaller with further aging, and disappear by 14 months.

This difference between the TG and WT mice is again illustrated in FIG. 3, which presents the data in two different ways. In 3A, the ABR thresholds were averaged across two separated frequency segments (2, 4 and 8 kHz as the low-frequency (LF) region (solid lines), and 16, 32, 48 and 64 kHz as the high-frequency (HF) region (dashed lines)). It appears that, the between-group difference in the high-frequency region is already apparent at 2 months of age in favor of the TG group. In the TG group, the averaged HF threshold slowly increases with age, roughly parallel with the WT group up to 6-8 months of age, and then stabilizes, whereas the HF thresholds in the WT group continue to deteriorate, albeit at a slightly slower rate than that seen from 2 to 8 months. This results in an increasing HF differential between the two groups. The averaged thresholds in the LF region are very close to each other between the two groups early in life (2 and 4 months). Later, the development of LF hearing loss is slower during the 4-8 months age period in TG group, resulting in a larger difference between the groups during this period. In contrast to the HF region, LFHL does not seem to stabilize in the TG group after 8 months, but rather continues to progress in a higher rate than in WT group. Therefore, LF hearing loss in the TG group catches up after 8 months of age and becomes closer to the values from the WT group by 14 months.

To further appreciate the protective effect of XIAP-Myc on hearing, the averaged ABR-threshold audiogram in WT group obtained at 6 months is compared with that from the TG groups at 14 months in FIG. 3B. Clearly, the thresholds at the three high-frequencies (16, 32 and 48 kHz) obtained at 14-month TG mice are better than those from 6 month WT mice, suggesting that the HF hearing loss was delayed by more than 8 months.

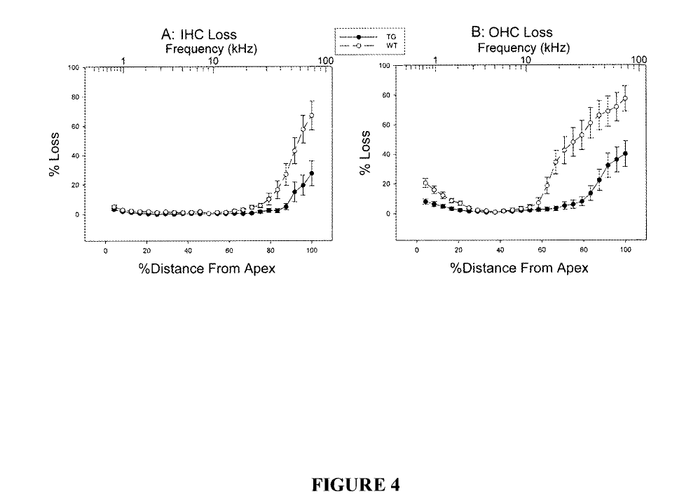

Hair cell loss was evaluated from 19 cochleae in each group. FIG. 4 compares the averaged losses of both IHCs (4A) and OHCs (4B) between the groups. Generally, the IHC loss is much less than OHC loss in the two groups and is only seen at the high-frequency end of the cochleae. In the WT group (right panel), the OHC loss is above 70% for the basal (HF) 10% end of the cochleae, spreading to the middle of the cochlea duct. In the TG group (Left panel), the OHC loss is less than 30% for the basal 10% end of the cochlea duct and the loss is restricted more to the high-frequency region. Putting the IHC and OHC losses together, the averaged hair cell loss in WT group is 665.47±417.99 cells per cochlea (mean±SD), while the value in TG group is 219.95±258.4 cells per cochlea. The difference is strongly significant (t-test, t=3.982, p<0.001).

FIG. 5 shows representative cochlea surface preparation images from two mice (two in the left panel from a TG mouse, and two in the right from a WT mouse). A smaller degree of OHC loss is seen in the TG cochlea at the very basal location (basal-1, 10% of the distance from the basal end) and the loss decreases when the as image is moved slightly towards the apex (basal-2, 10-20% of the distance from the basal end). Only scattered IHC loss is seen at the very basal end of the cochlea. In the two images taken from the WT cochlea at comparative locations, the OHC loss is much more severe. In addition, significant IHC loss is also seen in these two images of the WT cochlea (FIG. 5, right panel).

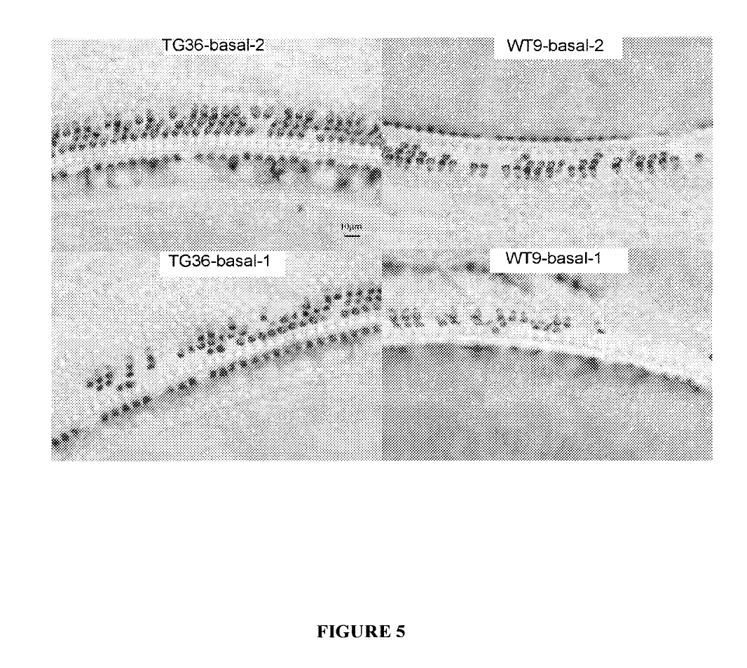

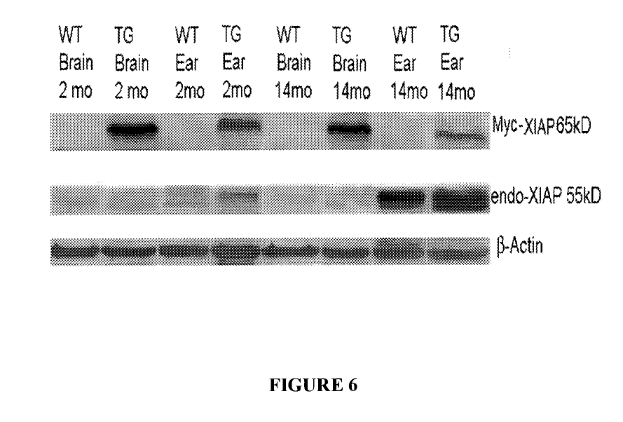

Western blot analysis was successful for 14 TG and 11 WT cochleae obtained from the mice that had been observed for 14 months and had completed their 14 month ABR. To explore further the effects of age on the expression of both endo-XIAP and XIAP-Myc, the same numbers of young mice (2 month old) in each genotype were tested. Therefore, the Western samples are divided into four groups according to ages and genotypes (young-TG, young-WT, old-TG, and old-WT). From each animal, the tissue from one cochlea and a piece of brain at the temporal lobe were used for detecting both XIAP-Myc and endo-XIAP. Relative levels of both XIAPs were calculated against the volume of β-actin to generate the volume ratio. To avoid confounding from potential technical variations across different gels, the samples were arranged, as indicated in FIG. 6, so that each gel contained 8 samples from both (2) brain tissue and ear tissue from all 4 groups (2×4). Interestingly, it was found that, while the XIAP-Myc expression seemed not change with age, the endo-XIAP appeared to increase in older ears (14 months) but not in the older brain. As shown in FIG. 7, the endo-XIAP appears to be higher in the ears than in the brain for both genotype groups. This difference is much higher in older mice. A 3-way ANOVA was performed to identify the impact of age (2 versus 14 months), genotype (WT versus TG) and tissue (brain versus cochlea) on the endo-XIAP levels. A significant effect was found for both age and tissue factors (p<0.001), but not for genotype factor (p=0.1). The null effect of genotype suggested that the transferred ub-xiap gene did not interfere with the expression of endo-xiap gene. Because of this, we analyzed the age effect by grouping the samples from both genotype groups stratified by age. Since there is no proven genotype effect on endo-XIAP level, cochleae and brain tissues from the two genotype groups are pooled for the analysis of age effect. Within the age factor, the endo-XIAP level was compared between the 25 old cochleae (from 14 month old mice after chronological ABR testing) and these from 2 month old mice of the same sample size. It appears that the endo-XIAP is much higher in the older cochlea (1.359±0.4 for 14 month cochleae versus 0.729±0.282 for 2 month-old cochleae (mean±SD), t=6.437, p<0.001). By contrast, a paired t-test failed to show a significant difference in levels of endo-XIAP in the brain for the two ages (0.525±0.178 for 2 months and 0.709±0.528 for 14 months, n=24 in each age group). Therefore, the significant age effect was largely due to the increase of endo-XIAP in the cochlea with age. Also, a significant tissue effect was indicated by the fact that the level of endo-XIAP was found to be higher in the cochlea than in the brain at both ages (t=2.921 and p=0.007 for the age of 2 months; t=4.354 and p<0.001 for the age of 14 months).

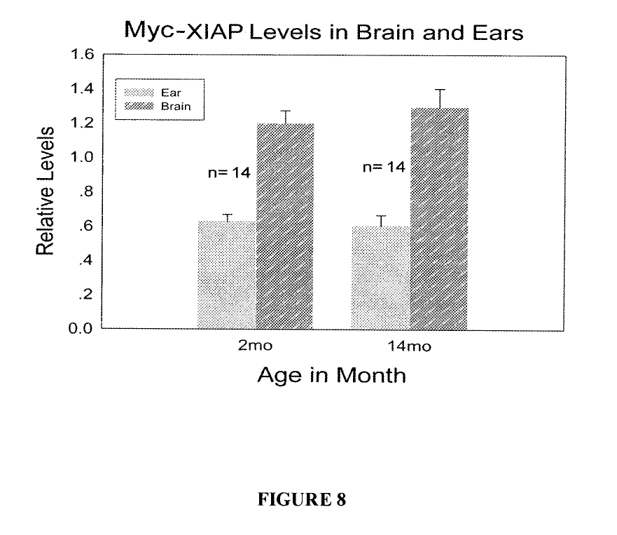

The level of XIAP-Myc appears to be independent of age, but different in the two types of tissues. FIG. 8 shows the expression of XIAP-Myc in both ears and brains for two age groups of TG mice. A 2-way ANOVA was performed against the two factors (age and tissue) that could potentially impact the level of XIAP-Myc. A significant effect of tissue was found (P<0.001). Unlike the endo-XIAP, the XIAP-Myc was found to be at a higher level in the brain than in the ear. For example, at 14 months, the XIAP-Myc is 0.601±0.24 (mean±SD) in the ear and 1.295±0.442 in the brain (t=6.621, p<0.001). However, the effect of age was not significant, suggesting that the transferred ub-xiap gene is expressed in a stable manner, which does not change with age.

II. Enzyme Facilitated Cochlear Gene Transfection with AAV

RWM becomes permeable to AAVs after digestion with the enzyme for a short period of time. Cochlear gene transfection is verified by immunostaining against GFP in the ears that are treated with the enzyme but not the untreated ear. FIG. 10 shows the transfection of a treated ear, the density of transfected cells is comparable with the ear transfected through cochleostomy. Successful transfection using this method was seen in both guinea pigs and rats.

The RWM treatment with the enzyme is functionally safe. The treatment does not cause significant hearing loss (less than 15 dB threshold shift was seen at the high end of the frequency range (32, 48 kHz), and no hearing loss at other frequencies tested. This conclusion is drawn from the results of auditory brainstem response (ABR) tests in 12 ears before and 2-4 weeks after the surgery.

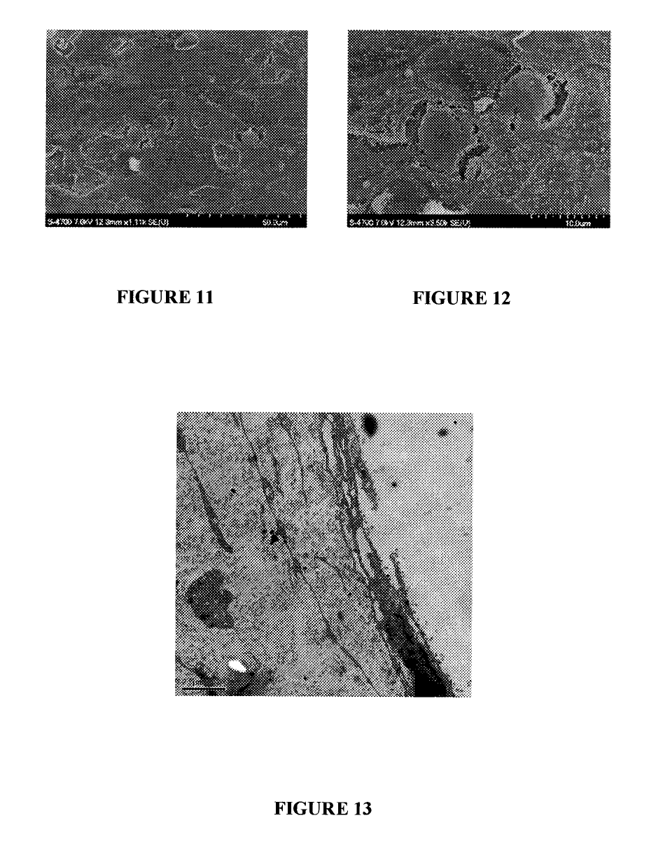

The enzyme treatment causes damage to the epithelia facing middle ear. Such damage can be seen in both SEM and TEM. FIGS. 11 and 12 show the SEM images of damaged RWM facing middle ear in two different magnitudes.

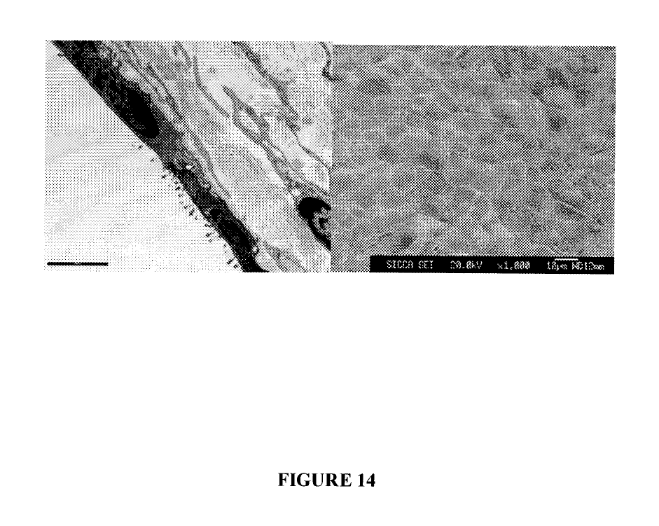

The damage to RWM by the enzyme is, however, temporary. Images of RWMs 3 weeks after the treatment show no difference from normal control. FIG. 14 shows the samples at this time point (Left: TEM, Right: SEM).

Animal models have been used previously as indicators of hearing loss in humans (see for example: Howard W. Francis et al. “The functional age of hearing loss in a mouse model of presbycusis. II. Neuroanatomical correlates.” Hearing Research 183 (2003)29-36; and Ohlemiller KK. “Mechanisms and genes in human strial presbycusis from animal models”. Brain Res. 2009 Jun. 24; 1277:70-83).

We found recently that the hearing sensitivity of 14 month ubXIAP mice was similar (or comparable) to that of 6 month WT mice. Hearing loss in ubXAIP mice at 6 months of age was delayed by 8 months, which is approximately ⅓ life span of this strain, which assume that a mouse lives on average for 18 months. Given that the average life expectancy for a human subject is about 80 years then we would expect to get about 25 more years of “normal” hearing using the techniques described hereinabove.

One skilled in the art can appreciate further features and advantages of the invention based on the above-described embodiments. Accordingly, the invention is not to be limited by what has been particularly shown and described, except as indicated by the appended claims.

1. A method of treating or preventing hearing loss in a subject, the method comprising: administering to the subject in need thereof, an adeno-associated viral expression vector encoding X-linked inhibitor of apoptosis protein (XIAP), the XIAP being positioned in the vector for expression in an inner ear organ, or associated neural structures, of the subject so as to treat or prevent the hearing loss.

2-8. (canceled)

9. A method of treating or preventing impaired balance in a subject, the method comprising: administering to the subject in need thereof an adeno-associated viral expression vector encoding X-linked inhibitor of apoptosis protein (XIAP), the XIAP being positioned in the vector for expression in a cell of the vestibular organ so as to treat or prevent the impaired balance.

10. (canceled)

11. The method, according to claim 1, in which the adeno-associated viral expression vector is selected from the group consisting of: AAV1, AAV2, AAV3, AAV4, AAV5, AAV6 and AAV7.

12. (canceled)

13. The method, according to claim 1, in which the adeno-associated viral expression vector is a modified serotype-2 or -8 AAV vector.

14. The method, according to claim 1, in which the XIAP is full length human XIAP.

15. The method according to claim 1, in which the hearing loss is high-frequency hearing loss is at 2 kHz and above.

16. The method, according to claim 1, in which the inner ear organ includes the inner ear hair cell and the outer ear hair cell.

17. The method, according to claim 16, in which the inner ear cell is a hair cell, a supporting inner ear cell, inner ear mechanical structure or a spiral ganglion neuron.

18. The method, according to claim 1, in which the associated neural structures are the efferent and afferent neural processes.

19. The method, according to claim 1, in which the hearing loss is the result of hair cell degeneration in the cochlea, or loss of supporting mechanisms that allow the hair cell to function.

20. The method, according to claim 1, in which the hearing loss is the result of spiral ganglion neuron degeneration in the cochlea.

21. The method, according to claim 1, in which the hearing loss is presbycusis.

22. (canceled)

23. The method, according to claim 1, in which the subject is human.

24. The method, according to claim 1 in which the vector is administered through the round window membrane

25. The method, according to claim 1, in which a ubiquitin promoter is used to drive expression of XIAP in cochlea cells.

26. The method, according to claim 1, in which the hearing loss is due to ototoxicity, noise induced hearing loss, viral infections of the inner ear, autoimmune inner ear diseases, genetic hearing losses, inner ear barotrauma; physical trauma, or surgical trauma, or inflammation.

27-34. (canceled)

35. A method of delivering a viral vector to an inner ear, the method comprising:
a) partially digesting the round window membrane using a proteolytic enzyme; and
b) contacting the partially digested round window membrane with an adeno-associated viral expression vector so as to transfect the inner ear cells or associated neural structures.

36. The method, according to claim 35, in which the adeno-associated viral expression vector is selected from the group consisting of: AAV1, AAV2, AAV3, AAV4, AAV5, AAV6 and AAV7.

37. (canceled)

38. The method, according to claim 35, in which the AAV is located for expression of X-linked inhibitor of apoptosis protein (XIAP), the XIAP being positioned in the vector for expression in an inner ear organ, or associated neural structures.

39. The method, according to claim 35, in which the adeno-associated viral expression vector is a modified serotype-2 or -8 AAV vector.

40. The method, according to claim 35, in which the proteolytic enzyme is a collagenase.

41. The method, according to claim 40, in which the collagenase is collagenase from Clostridium histolyticum, Type II.

42. The method, according to claim 40, in which the enzyme is used to partially digest the round window membrane at a concentration of between 50 and 200 unit/ml.

43. (canceled)
